(19) United States
(12) Patent Application Publication
(10) Pub. No.: US 2025/0204784 A1
(43) Pub. Date: Jun. 26, 2025

(54) METHOD FOR ACQUIRING IMAGE DATA USING PILOT TONE

(71) Applicant: Siemens Healthineers AG, Forchheim (DE)

(72) Inventors: Peter Speier, Erlangen (DE); Yan Tu Huang, Shenzhen (CN); Mario Bacher, Diepersdorf (DE)

(21) Appl. No.: 18/991,688

(22) Filed: Dec. 22, 2024

(30) Foreign Application Priority Data
Dec. 22, 2023 (EP) 23219863.0

(51) Int. Cl. A61B 5/00; A61B 5/055

(52) U.S. Cl. CPC A61B 5/0044; A61B 5/055; A61B 5/7267; A61B 2576/023

(57) ABSTRACT

A method for acquiring image data from a part of a human body subjected to a cardiac movement is provided. The method includes transmitting a radiofrequency Tx Pilot Tone signal, receiving a pilot tone signal including a number of channel signals, and carrying out a blind source separation algorithm on a training portion of the pilot tone signal and thereby determining weighting vectors. The method also includes selecting and storing at least two non-parallel weighting vectors that allow to extract signal components from the number of channel signals. The extracted signal components represent cardiac movement. The method includes applying the weighting vectors to the further portions of the pilot tone signal to obtain a multi-dimensional pilot tone signal representing the cardiac movement, and using the multi-dimensional pilot tone signal for triggering the acquisition of the image data.

This application claims the benefit of European Patent Application No. EP 23219863.0, filed on Dec. 22, 2023, which is hereby incorporated by reference in its entirety.

### BACKGROUND

The present embodiments relate to a method for acquiring image data in a

radiological examination of a part of the human or animal body using a Pilot Tone method, a computer program, and a control unit for controlling a radiological imaging system, such as a magnetic resonance imaging system.

Independent of the grammatical term usage, individuals with male, female, or other gender identities are included within the term.

Patient movement or motion during a diagnostic examination or scan of medical data (e.g., during radiological imaging) may cause artifacts in the acquired images. Especially Magnetic Resonance (MR) imaging is relatively slow, so that cyclical motions such as respiration and cardiac movement, but also patient motion with respect to the measurement system, may occur during the scan. If the movement is known, the data acquisition may be triggered to a particular phase in the cyclical movement, or the acquired data may be corrected.

It is therefore known to acquire an electrocardiogram (ECG) of the patient during radiological imaging in order to trigger the data acquisition to a particular phase in the cardiac cycle. However, taking an ECG during a magnetic resonance imaging (MRI) scan presents difficulties (e.g., because of the high magnetic fields that may cause interferences in the ECG leads). Other methods for motion detection include breathing belts, the use of two-dimensional (2D) or three-dimensional (3D) cameras, radar, and radiofrequency (RF) based methods.

The basic principle of RF signal based methods optimized for use in MRI, also termed “Pilot Tone” methods, is to irradiate the body with an RF signal just outside the receive bandwidth of the MR data being acquired, but within the frequency range of the MR receive system (e.g., within the oversampling bandwidth that is acquired during every readout). To transmit the RF signal, an RF transmitter may be installed in the radiological imaging modality (e.g., above the patient). The transmitted Pilot Tone signal interacts with the human body and is received via multiple antennas (e.g., the local RF coil(s)). The received multi-channel Pilot Tone signal may be analyzed in order to extract specific motion components. For example, Principal Component Analysis (PCA) techniques may be used, as disclosed in EP3413076A1, in order to separate orthogonal signal components from the multi-channel Pilot Tone signal. Generally, one signal component is extracted for each movement type (e.g., for the cardiac movement).

The advantage of RF based methods for detecting patient motion during magnetic resonance imaging (MRI) scans is that the RF transmission and reception infrastructure is already present.

In principle, the Pilot Tone method may be used for respiratory and cardiac triggering and gating. The cardiac application is especially challenging because the modulation of the Pilot Tone signal caused by the cardiac motion is rather small (e.g., compared to the respiratory signal). In case the Pilot Tone method is used during an MR acquisition, the Pilot Tone signal may be affected by RF pulses, thereby compromising trigger stability.

Another limitation of cardiac Pilot Tone methods is that the cardiac signal does not show a clear R-wave, which may be used as a trigger time point. Generally, choices of trigger time points are limited to periods where the signal is changing significantly. If it remains constant or the change is so small that it cannot be reliably detected over a period of time, no trigger may be placed in this period. Therefore, current Pilot Tone implementations trigger well after the start of the cardiac contraction, using a trigger time point in the cardiac cycle approx. 200 ms after the R-wave in the ECG. This may reduce stability (e.g., in MR acquisitions that are preceded by a “dark blood” double inversion magnetization preparation), which is to be played out before the contraction, so that one needs to trigger the preparation before the start of contraction of the heart muscle during systole.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the above-identified limitations of Pilot Tone methods on the trigger stability and on the possible trigger time points are improved.

According to a first aspect, a method for acquiring image data in a radiological examination of a part of the human or animal body is provided. The part is subjected to a cardiac movement. The method includes the steps: transmitting a radiofrequency Tx Pilot Tone signal; receiving a Pilot Tone signal from the body part via a radiofrequency receiver coil arrangement including a number of (e.g., several) channels, where the received Pilot Tone signal includes a number of (e.g., several) channel signals associated with the several channels; carrying out a blind source separation algorithm on a training portion of the Pilot Tone signal and thereby determining weighting vectors to extract cardiac movement signals in the presence of other signals, where the weighting vectors allow to form weighted combinations of the several channel signals; selecting and storing at least two non-parallel weighting vectors that allow to extract signal components that represent cardiac movement, from the several channel signals; applying the weighting vectors to the further portions of the Pilot Tone signal to obtain a multi-dimensional Pilot Tone signal representing the cardiac movement, where the multi-dimensional Pilot Tone signal has at least two dimensions; and using the multi-dimensional Pilot Tone signal for controlling (e.g., triggering) the acquisition of the image data, or for retrospectively gating or correcting the acquired image data.

The present embodiments have realized that the cardiac signature in the Pilot Tone signal is not limited to a single component. Instead, at least two signal components contribute to the cardiac modulation. However, this is not reflected in prior art methods using a Pilot Tone method to trigger the acquisition of image data. Rather, so far, only the strongest signal component representing cardiac movement was used, whereas the other component or components were ignored. The present embodiments therefore propose to improve the quality of the Pilot Tone signal used for controlling the acquisition, as well as the trigger stability, by extending the dimensionality of the cardiac Pilot Tone signal from a one-dimensional signal to a multi-dimensional signal and utilizing these additional dimensions in the trigger detection. The number of dimensions is herein referred to as N.

The method of the present embodiments is carried out during the acquisition of image data in a radiological examination of a part of the human or animal body, where the part is subjected to cardiac movement. The multi-dimensional Pilot Tone signal obtained by the method of the present embodiments may be used to control the image data acquisition (e.g., for triggering the acquisition to particular phases in the cardiac cycle). Alternatively, the multi-dimensional Pilot Tone signal may be used to retrospectively gate or correct the acquired image data. For example, if the acquired image data may be assigned to particular cardiac phases, the image may be reconstructed using retrospective motion correction techniques.

The radiological examination may be any imaging examination using a medical imaging modality (e.g., computed tomography (CT), x-ray imaging, ultrasound imaging, or positron emission spectroscopy). In one embodiment, the radiological examination is a magnetic resonance imaging examination. This is advantageous because the RF infrastructure required for transmitting and receiving the Pilot Tone signal is already present. The Pilot Tone signal may be transmitted and acquired during the entire imaging procedure (e.g., during an MR scan) or at least during relevant parts thereof. The part of the human or animal body, of which image is to be acquired, is subjected to cardiac movement. The part may be, for example, the heart, the lungs, the brain, peripheral blood vessels, an organ within the abdomen, thorax, or any organ affected by a pulsating blood.

The method includes transmitting an RF transmit (Tx) Pilot Tone signal (e.g., via at least one RF transmit antenna, also referred to as PT generator). In one embodiment, the RF transmit antenna includes a magnetic near field generating loop. The Tx Pilot Tone (PT) signal may be a continuous wave (CW) signal and may be generated by an independent continuous wave RF source. The Tx Pilot Tone (PT) signal may also be a pulsed signal. In one embodiment, the frequency of the Tx PT signal is still within the bandwidth of the MR image acquisition, if the method is applied during an MR examination, to avoid interference with the MRI data. The frequency of the transmitted Pilot Tone signal may be between about 20 and 2000 kHz (e.g., between 200 and 1000 kHz or between 500 and 1000 kHz away from the center frequency of the MR signal, such as the MR signal acquired in the MRI examination). The Pilot Tone signal interacts with the body part, and the received PT signal is therefore modulated by its movement.

The RF receiver coil arrangement includes a number of (e.g., several) channels, as is common in MR receiver coil arrangements (e.g., local coils and coil arrays). Each element acquires a separate channel signal. Thus, the received Pilot Tone signal includes a number of (e.g., several) channel signals associated with the several channels of the RF receiver coil arrangement. In one embodiment, 4 to 8 or 8 to 64 channels are used. Different types of movement may affect the channels to different degrees, depending on the orientation and position of the moving body part with respect to the coil element and to the PT generator. The number of channel signals are thus to be combined in an intelligent way in order to extract particular movement types such as cardiac movement and/or respiratory movement.

The method of this present embodiments is directed to extract the cardiac movement, which provides that any movement caused by the cyclical contraction of the heart muscles, be it in the heart itself or in other body parts affected by the heart (e.g., by arterial blood vessels).

In order to extract the signal components pertaining to the cardiac movement, a blind source separation (BSS) algorithm is carried out on a training portion of the Pilot Tone signal. The extracted signal components may be independent, where “independent” is to be understood broadly as a general term to describe components found by any BSS method, including Principal Component Analysis (PCA). “Independent” is not to be understood as limited to components found by Independent Component Analysis (ICA). The principle of blind source separation (BSS) is the separation of a set of source signals from a set of mixed input signals, without or with minimal aid of information about the source signals or the mixing process. BSS aims at finding an independent set of vectors (herein also termed “weighting vectors”), onto which the input signals may be transformed. The data that is projected or mapped onto each vector corresponds to an independent source. Since the method involves projecting the data onto a set of axes/vectors that are determined by the nature of the data, the method is termed blind source separation (e.g., “blind” because the projection vectors are determined without of the use of any prior knowledge of the data structure). The BSS algorithm is applied on a training portion of the Pilot Tone signal. The training portion may be a short signal portion covering a few (e.g., one to twenty) heartbeats acquired before or at the beginning of the radiological examination. Alternatively, for example, when the Pilot Tone signal is to be applied retrospectively, the training portion may cover the complete or a part of the Pilot Tone signal acquired during the examination.

The BSS algorithm is thus used to determine weighting vectors in order to extract cardiac movement signals in the presence of other signals, where the weighting vectors allow to form weighted combinations of the several channel signals. The weighting vectors are nonparallel, which provides that the weighting vectors allow to extract different signal components (e.g., independent signal components), where the measure of independence may be defined in various ways, according to different embodiments. In an embodiment, the nonparallel weighting vectors allow to extract statistically independent signal components. Example techniques used for determining the weighting vectors will be explained below in more detail.

According to the present embodiments, at least two nonparallel weighting vectors are selected and stored. The at least two nonparallel weighting vectors allow to extract at least N=2 signal components that represent cardiac movement from the several channel signals. Thus, according to the present embodiments, not one, but at least two (e.g., independent) signal components are used to describe the cardiac movement. Once the weighting vectors have been determined from the training portion, the weighting vectors are applied to the further portions of the Pilot Tone signal to obtain the multi-dimensional Pilot Tone signal representing the cardiac movement. This signal is used for controlling (e.g., triggering) the acquisition of the image data, or for retrospectively gating or correcting the acquired image data. Thereby, higher dimensional components are added to the Pilot Tone signal and may be used for triggering the image acquisition. This has the advantage that the usage of the input signal is maximized, compared to the prior art approach, where the energy in the higher components is not used. This will allow to reduce the amplitude of the transmitted Pilot Tone signal without loss of stability, and reduce the likelihood of signal to noise ratio loss due to activation of the compander architecture in the receiver. Alternatively, trigger stability with respect to noise and RF artefacts may be increased for a given PT amplitude.

In addition, it has been found that the higher dimensional signal components show activity at different time points compared to the first (strongest) component. This will enable to extract new trigger time points in the cardiac interval from the PT signal. Especially, trigger time points earlier in the cardiac cycle (e.g., closer and even before the R-wave) will be possible. This reduces the prior art problems specifically when using MR sequences requiring magnetization preparation, such as for dark blood imaging. The present embodiments thereby allow to improve on a promise of the Pilot Tone method (e.g., should enable multiple trigger points in the cardiac cycle, such as at end systole or early diastole). The Pilot Tone signal has been found to follow the cardiac volume curve closely without any lag, except processing delays. However, with only a single signal component, the only practical prospective trigger time points for typical cardiac Pilot Tone signals were during mid and end systole or early diastole. By including further higher order signal components into the signal analysis, it is possible to extract many more useful trigger time points within the cardiac cycle, especially ones close or even preceding the R-wave.

According to an embodiment, the multi-dimensional Pilot Tone signal representing the cardiac movement has between two and five (e.g., two or three) dimensions (e.g., N=2-5 or 2-3.

With current PT signal strengths, it is believed that up to N=5 signal components may maximally be used for cardiac triggering. As the higher dimensional signal components will likely have lower energy, it is unlikely that any higher order components will be valuable. In one embodiment, the strongest 2 or 3 signal components will be extracted using the weighting vectors; thus, the multi-dimensional signal will be 2- or 3-dimensional.

According to an embodiment, the blind source separation algorithm may use techniques such as Principal Component Analysis (PCA), using, for example, singular value decomposition, or Independent Component Analysis (ICA). In both PCA and ICA, one attempts to find a set of axes or weighting vectors that are independent of one another in some sense. One assumes that there are a set of independent sources in the data, but does not assume their exact properties. By defining some measure of independence, it is then possible to decorrelate the data by maximizing this measure between projections onto each axis of the new space, into which the data is transformed. Thus, the independent signal components (e.g., which represent independent movement types) are obtained by applying the weighting vectors onto the received Pilot Tone channel signals.

According to an embodiment, the blind source separation algorithm utilizes one or more Principal Component Analysis (PCA) operations. In PCA, the optimized variable is the variance. By maximizing variance, PCA operations lead to a set of orthogonal signal components. PCA operations are useful, for example, to reduce the complexity of the BSS problem. For example, a dimensionality reduction may be performed by applying a PCA based on either eigenvalue or singular value decomposition, and using only the largest principle component's singular values.

For example, a PCA may be used to remove the respiratory signal component from the Pilot Tone signal. This may, for example, be performed as follows.

The received Pilot Tone signal is optionally pre-processed by low-pass or bandpass

filtering in order to avoid aliasing of high-frequency noise. This may optionally be followed by down-sampling (e.g., during portions of the PT signal acquired outside MT measurements). During MR measurements, a high sample rate may be maintained because the signal is frequently corrupted by the RF pulses of the MR measurement. The pre-processing is optional because it increases the signal to noise ratio but at the cost of additional time delay. Further, the pre-processed signal may be subjected to a normalization step, in which the phases of all channels are normalized to a reference phase. The phase normalization may be achieved by multiplying with the complex conjugate of the reference channel (e.g., one of the channels is selected as the reference channel).

According to this embodiment, the (optionally normalized) Pilot Tone signal is further processed to reduce all other signals but the respiratory one. This may be achieved by first bandpass filtering the signal to the respiratory frequency range, and then applying a PCA operation, in which the strongest principle components are extracted. The first few strongest components represent respiratory motion, and this part of the input signal (e.g., the received Pilot Tone signal) is then removed. On the remaining signal, an appropriate BSS algorithm (e.g., PCA or ICA) is repeated for the cardiac movement (e.g., on data filtered to the cardiac frequency range). Thereby, the weighting vectors that extract the cardiac movement signals may be determined. According to the present embodiments, at least 2 signal components representing cardiac movement are extracted. For example, the real part and the imaginary part of the first component (e.g., the component corresponding to the largest eigenvalue) may be extracted as the first signal component and second signal component, respectively.

According to an embodiment, the blind source separation algorithm utilizes one or more Independent Component Analysis (ICA) operations. ICA is based on the assumption that the individual signal components are statistically independent from each other. Thus, ICA operations find the independent components by maximizing a measure of statistical independence of estimated components. The measure of independence between the axes may, for example, be based on non-Gaussianity, and the axes are not necessarily orthogonal. Thus, solving the ICA problem may be formulated as a problem of, for example, minimizing the mutual information or maximizing the non-Gaussianity. Gradient descent methods or other optimization techniques may be used.

According to an embodiment, the blind source separation algorithm calculates a demixing matrix, where the demixing matrix extracts independent (e.g., statistically independent) components from the several channel signals. Whatever BSS analysis method is used (e.g., PCA or ICA), it is useful to calculate a demixing matrix. The demixing matrix may be applied to the further portions of the Pilot Tone signal in order to extract the independent signal components from the several channel signals of the received and optionally pre-processed Pilot Tone signal. Depending on the implementation, the demixing matrix may be either complex or real valued. In practice, it is sufficient to store and apply to the subsequent data only the parts of the demixing matrix that are necessary to calculate the desired components.

According to an embodiment, the blind source separation algorithm is used to detect the strongest independent component corresponding to the cardiac movement, and the method includes using the strongest independent component to retrospectively analyze the training portion of the Pilot Tone signal (e.g., to average the Pilot Tone signal over a plurality of cardiac intervals, the cardiac intervals having been determined from the strongest statistically independent component), and detecting at least one further independent component corresponding to the cardiac movement from the retrospective analysis.

This embodiment is highly useful, because usually the strongest independent component corresponding to the cardiac movement may be reliably detected. The higher order components (e.g., the second, third, . . . strongest signal components) are harder to detect, as their energy is lower. However, it is known that they have exactly the same (varying) periodicity as the first, strongest component. Therefore, one may stabilize their detection after retrospective trigger detection on the first component. This may, for example, be done by averaging the received Pilot Tone signal over a plurality of cardiac intervals. For example, the averaging is done over the exact cardiac intervals, the length of which vary over time, and which have been determined from the strongest independent component. After the averaging, the signal may be subjected to another PCA or ICA operation in order to detect at least one further independent component corresponding to the cardiac movement from the averaged data. Other forms of retrospective analysis may consist in removing the strongest independent signal component from the data.

According to an embodiment, the channel signals of the received Pilot Tone signal are complex-valued, and the weighting vectors each extract a real or an imaginary part of an independent signal component.

Since the RF coil may also detect the phase, the several channel signals may be complex valued. It is useful to treat real and imaginary parts as separate signal components, since the real and imaginary parts may in fact contain independent information from one another. Therefore, the weighting vectors may each extract either a real or an imaginary part of an independent signal component, making this real or imaginary part one separate signal component that may be used in the multi-dimensional Pilot Tone signal. In other words, a signal component having a real and imaginary part is a two-dimensional Pilot Tone signal for the purposes of the present embodiments. A real and imaginary part must not necessarily belong to the same independent signal component, as extracted, for example, by ICA or PCA. It is also possible that the multi-dimensional Pilot Tone signal includes two real parts or two imaginary parts of different independent signal components.

According to an embodiment, the channel signals of the received Pilot Tone signal are complex-valued. The channel signals are rotated in the complex plane before carrying out the blind source separation algorithm (e.g., rotated so that a mean of the rotated signal lies on one of the diagonals of the complex plane). This embodiment is advantageous because the real and imaginary components thereby become equally strong, making the channel signals numerically easier to process (e.g., by PCA or ICA). For example, the channel signals may be rotated in the complex plane so that a mean of the rotated signal is on one of the diagonals of the complex plane. In other words, abs(real(PT))=abs (imag(PT)).

According to an embodiment, the channel signals of the received Pilot Tone signal are complex, and the blind source separation algorithm is real-valued. The method includes generating a real-valued matrix in which the real and imaginary parts of the complex channel signals form separate channels, and performing a blind source separation algorithm on the real-valued matrix.

As stated above, that the two or three strongest cardiac components are captured in the complex-valued strongest signal component is a special case. In general, the two or three cardiac components of relevance are distributed over two principal components. Therefore, one needs a way to separate and capture the first 2 to 5 or 2 to 3 cardiac signal components in all cases. This may be done in a number of (e.g., several) ways. In one embodiment, the complex valued DSS is substituted by a real valued BSS (e.g., composite real valued PCA), where the real and imaginary parts of the complex channel signals are used to generate a real valued matrix without information loss (e.g., by forming the magnitude). Thus, if the acquired PT signal has the size (e.g., channels, samples), then the real valued matrix has the size (e.g., 2* channels, samples). This may be done by concatenating the real and imaginary parts of the channel signals into the real valued matrix. In a next step, a real valued BSS algorithm (e.g., using PCA operations) is performed on the real valued matrix. The 1st N (e.g., 2 to 5 or 2 to 3) strongest signal components (e.g., principal components or statistically independent components) are stored as weighting vectors. Thus, the result of applying these weighting vectors is a N-dimensional signal for each time point. This N-dimensional PT signal may also be referred to as “vector cardiac Pilot Tone” signal or VCPT. The VCPT signal may be used to stabilize the signal by using more input data (e.g., signal components), and suppress and reject artefacts. In one embodiment, the artifacts from RF pulses may be better detected and suppressed. Further, the detection of trigger points may be stabilized because it may be based on multi-dimensional features, and not on a single feature of a one-dimensional signal. Because of these advantages, the VCPT will enable other trigger time points, especially an earlier one, thus improving robustness of dark blood and tagging measurements.

According to an embodiment, trigger time points for triggering the acquisition of the image data are determined by evaluating properties of the multi-dimensional Pilot Tone signal or of the time derivative of the multi-dimensional Pilot Tone signal (e.g., by evaluating one or more of its position, direction, velocity, acceleration, or change in direction in the multi-dimensional signal space).

The multi-dimensional Pilot Tone signal representing the cyclical cardiac movement may be represented in a multi-dimensional space having N dimensions. Since the cardiac movement is cyclical, the signal will move in the n-dimensional space in closed curves. For example, if the Pilot Tone signal is two-dimensional, it may, for example, be represented by a real component and an imaginary component, which may be plotted in the complex plane over multiple cardiac cycles. If there was only one, real valued component containing the Pilot Tone signal, the signal would have a linear shape, oriented in an arbitrary direction in the plane. However, in the complex plane, the signal displays a repetitive two-dimensional pattern, with characteristic features that may be used as trigger points. By retrospectively analyzing such a multi-dimensional plot, one may derive suitable characteristic features from the n-dimensional signal pattern, which may serve as trigger time points for triggering the acquisition of the image data. These characteristic features may be detected practically in real time when acquiring further portions of the Pilot Tone signal. In real-time provides that the trigger time point is only delayed by the necessary data transfer delays and signal processing delays (e.g., by group delays of applied low pass filters).

Instead of the n-dimensional Pilot Tone signal itself, one may also analyze the time derivative. It has been demonstrated that the time derivative displays a particularly distinguishable feature at the end of the diastole. Therefore, according to an embodiment, the time derivative of the multi-dimensional Pilot Tone signal is used for controlling (e.g., triggering) the acquisition of the image data.

Thus, gating or trigger time points may be determined by evaluating properties of the n-dimensional Pilot Tone signal or of the time derivative of the n-dimensional Pilot Tone signal. Both of these signals may be plotted in n-dimensional space. The gating or trigger time points may be derived by evaluating one or more of the signal's position, direction, velocity, acceleration, or change in direction in the multi-dimensional signal space. For example, the image acquisition may be triggered each time the n-dimensional Pilot Tone signal travels through a defined region in the n-dimensional space. Alternatively, it may be triggered by an acceleration phase (e.g., when the acceleration follows a phase of slow motion through the signal space). This would, for example, indicate the beginning of contraction after the diastole. The trigger time points may also be determined by evaluating two different properties of the n-dimensional Pilot Tone signal (e.g., position and direction, position and change in direction, or position and acceleration). For example, trigger time point may be determined when the signal experiences a certain acceleration or change in direction when within a certain n-dimensional area of the n-dimensional signal space.

According to an embodiment, the acquisition of the image data is triggered at a defined time point within the cardiac cycle (e.g., at a time point between 250 ms before and 50 ms after an R-wave, between 200 ms before the R-wave and the R-wave, or between 150 ms and 20 ms before an R-wave). As explained above, with the method of the present embodiments, it is possible to detect a trigger time point even before the R-waves. It is hypothesized that the detected property of the n-dimensional PT signal is caused by the contraction of one atrium or both atria. It has been found that the method of the present embodiments is sensitive enough to detect a time point even before the R-wave and therefore a very useful trigger time point. Triggering before the R-wave is highly useful, since it allows to start the acquisition (e.g., magnetization preparation pulses) even before the ventricle is contract.

According to an embodiment, the method includes a further step of applying an adaptive, stochastic, or model-based filter to the multi-dimensional Pilot Tone signal representing the cardiac movement, to obtain a filtered movement signal. The filter may determine, in a stable way, one or more of derivatives of the signal (e.g., its first derivative, velocity). The Filter may be, for example, a Kalman filter, an extended Kalman filter, or a switched Kalman Filter. Possible filtering methods are disclosed in EP3413076 A1 and are incorporated herein by reference.

If the filtered movement signal is to be used in triggering, the filter may not introduce significant delay. Therefore, advanced filters such as adaptive, stochastic, or model-based filters may be provided. In embodiments, the filter not only denoises the movement signal, but already assigns segments or specific points of the movement signal to the respective phases of the cardiac movement. In one embodiment, the adaptive, stochastic, or model-based filter is first trained or adapted to the multi-dimensional PT signal derived from the training portion. Thus, the training portion of the Pilot Tone signal may be used also to configure the filter, since the filter may, during this training phase, generate or adapt a model of the movement signal to the actual Pilot Tone signal acquired in that particular measurement.

In other words, segmentation of the cardiac component enables triggering, for example, on the start/end of distinct cardiac phases. In addition, model-based segmentation is robust against measurement noise and may enable triggering on any, arbitrary points in the cardiac cycle. In the absence of severe arrhythmia, model-based filtering methods may also be able to predict cardiac activity beyond the current cardiac phase.

In one embodiment, the filter is a Kalman Filter, an Extended Kalman filter, or a Switched/Switching Kalman Filter. The Switching Kalman Filter switches between a number of (e.g., several) models during various phases of the cyclical movement. Kalman filtering, also known as linear quadratic estimation (LQE), is an algorithm that uses a series of measurements observed over time, containing statistical noise and other inaccuracies, and produces estimates of unknown variables that tend to be more accurate than those based on a single measurement alone, by using Bayesian inference and estimating a joint probability distribution over the variables for each timeframe. Thus, the Kalman filter provides, based on the past measurements (e.g., the training portion), for each filtered data point a probably correct data point. The Switched Kalman filter may also include information on the physiological phase of the data point (e.g., may already perform segmentation). The Kalman, Extended Kalman and Switched/Switching Kalman filter make use of prior information trained on actual data. Thus, these and other model-based filters make use of a model of the movement signal. Once such a model has been generated (e.g., by analysis of the cardiac component trace acquired during the training phase), segmentation may be achieved by various methods, such as Hidden Markov Models or Switched Kalman Filters. These methods may also be used retrospectively to obtain segmentations of the cardiac component.

As described above, in embodiments, the adaptive, stochastic, or model-based filter automatically segments the N-dimensional PT signal into two or more sections corresponding to two or more physiological phases of the cyclical movement (e.g., to the phases of the cardiac movement such as systole and diastole).

Accordingly, in embodiments, the time points used for triggering a scan of medical data from the part of the human or animal body, or for post-processing a scan of medical data performed during the acquisition of the Pilot Tone signal, are extracted from the filtered n-dimensional PT signal. In one embodiment, the time points are based on properties of the curve or on parameters of the stochastic or adaptive or model-based filter. In embodiments, the time points used for triggering or post-processing may be directly derived from the stochastic, adaptive, or model-based filter, once it has been trained.

According to an embodiment, the adaptive, stochastic, or model-based filter is adapted to obtain properties of the multi-dimensional Pilot Tone signal. The properties of the multi-dimensional Pilot Tone signal are, for example, a velocity vector or acceleration vector. As explained above, it has been found that the velocity and/or acceleration vector provides significant information that may be used to find the desired early trigger time point.

The present embodiments are further directed to a computer program including program code that induces a control unit of a radiological imaging modality to perform the method of the present embodiments when the program code is executed on the control unit. In one embodiment, the computer program may be started and ended by a user.

The present embodiments are further directed to a digital storage medium (e.g., a non-transitory computer-readable storage medium) including the program code as described. The digital storage medium may be in the form of a hard drive, such as a magnetic hard disk or a solid state drive, or in the form of a portable storage medium such as a CD, DVD, or USB-Stick, or in the form of a network storage medium, such as a NAS-storage or a cloud storage.

The present embodiments are further directed to a control unit adapted for performing the method as described, where the control unit may be part of a radiological imaging modality (e.g., a magnetic resonance system).

The present embodiments are further directed to a radiological imaging modality (e.g., a magnetic resonance system) including such a control unit. The magnetic resonance system is configured for performing the method of the present embodiments.

### BRIEF DESCRIPTION OF THE DRAWINGS

FIG. 1 shows a schematic flow diagram of an embodiment of a method.

FIG. 2 shows an example two-dimensional derivative of a cardiac PT signal in the complex plane.

FIG. 3 shows an MR-scanner in a schematic view.

### DETAILED DESCRIPTION

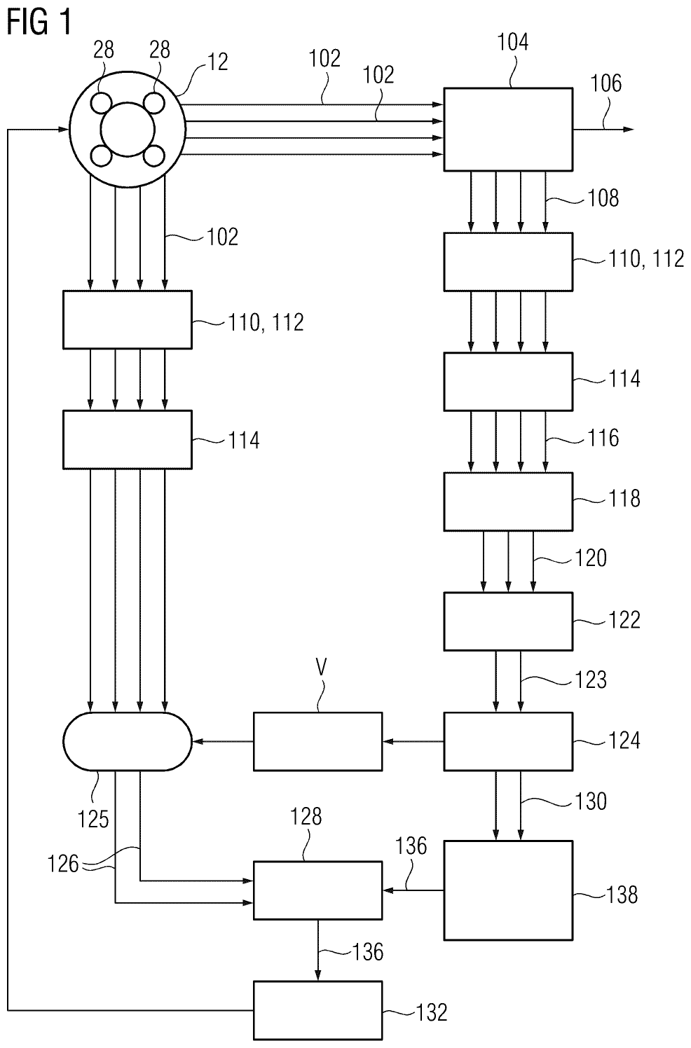

In the following and with reference to FIG. 1, an embodiment of a method is provided. In the example, a Pilot Tone (PT) signal is acquired during a magnetic resonance (MR) scan. An MR scanner 12 including a receiver coil arrangement 28 with (in this schematic example) four coils/channels is shown on the top left. The MR scanner 12 also has a Tx RF antenna for transmitting the PT signal. The receiver coil arrangement 28 acquires a signal 102 having 4 channel signals. The signal 102 is first subjected to a calibration step 104. The purpose of the calibration step 104 is to separate the MR imaging signal from the additional Pilot Tone signal. The MR data 106 is further processed to produce MR image data, as is known in the art.

The Pilot Tone signal 108 including the 4 channel signals is optionally pre-processed by low-pass or bandpass filtering 110 (e.g., to avoid aliasing of high-frequency noise), followed by down-sampling 112. This is because the MR signal is acquired at a very high sampling rate, which is not required for the analysis of cardiac motion. The pre-processing is optional because it increases signal to noise ratio (SNR) but at the cost of additional time delay.

The pre-processed signal may further be subjected to a normalization step 114, in which the phases of all channels are normalized to a reference phase. The phase normalization may be achieved by multiplying with the complex conjugate of the reference channel (e.g., one of the channels is selected as the reference channel).

The normalized, complex Pilot Tone channel signals 116 are then further processed to separate the various motion components modulating the Pilot Tone signal. This is done first by Principle Component Analysis 118, in which the largest principle components 120 corresponding to respiratory movement are extracted, as described above. The largest principle components 120 corresponding to respiratory movement are then removed from the channel signals, and the respiration-free channel signals are subjected to a further BSS algorithm (e.g., an Independent Component Analysis 122). Through the ICA, the strongest N independent signal components 123 corresponding to the cardiac movement are identified and separated. In one embodiment, the number N is predefined. The corresponding weighting vectors V for each independent signal component corresponding to the cardiac movement are calculated in step 124. The step 124 may be done automatically (e.g., by calculating the signal energy in the cardiac motion band for each independent component, compared to the signal energy in other frequency bands, and selecting the components with the highest relative signal energy in the cardiac motion band). Alternatively, the degree of correlation of each signal component with a typical cardiac component trace may be calculated. Once the strongest N independent components representing the cardiac motion have been selected, the weighting vectors V may be automatically calculated. The weighting vectors V correspond to a linear combination of the channel signals 102/108 of the number of receiver channels. The weighting vectors V are applied to the training portion of the PT signal to obtain the VCPT signal portion 130. The time derivative of the VCPT signal is then determined (e.g., by a constant velocity Kalman filter). The further processing (e.g., the detection of trigger points) is carried out on the derivative. Thus, the time derivative of the VCPT signal portion 130 is analyzed in step 138 to determine suitable trigger time points 136. For example, the time derivative of the VCPT signal 130 may be averaged over a number of (e.g., several) heart cycles and searched for characteristic features, such as an acceleration or change in direction in the n-dimensional signal space. This feature is then used in step 132 to detect the trigger time point on the incoming further Pilot tone signal data 102.

The weighting vectors V are stored and applied to the incoming further Pilot Tone signal data 102. In some embodiments, the incoming data 102 may first be subjected to low-pass filtering, extrapolation across invalid samples (e.g., during RF pulses), down-sampling 110/112, phase-normalization 114, or any combination thereof. The normalized complex channel signals are then multiplied with the weighting vectors V in step 125 to obtain the N-dimensional Pilot Tone signal 126 corresponding to the cardiac movement.

The N-dimensional Pilot Tone signal 126 may be subjected to a model-based filter 128 as described above. In some embodiments, the filter 128 is first trained on a training portion of the PT signal. The above-described adaptive filters such as the Kalman Filters and Switched Kalman Filters need some time to converge; thus, a calibration is useful to provide fast convergence, but not absolutely necessary. In other applications, the filter 128 adapts over time to the incoming movement signal 126 and does not require a calibration.

In the step 128, the trigger time points are detected and used to trigger the MR acquisition in the MR scanner 12.

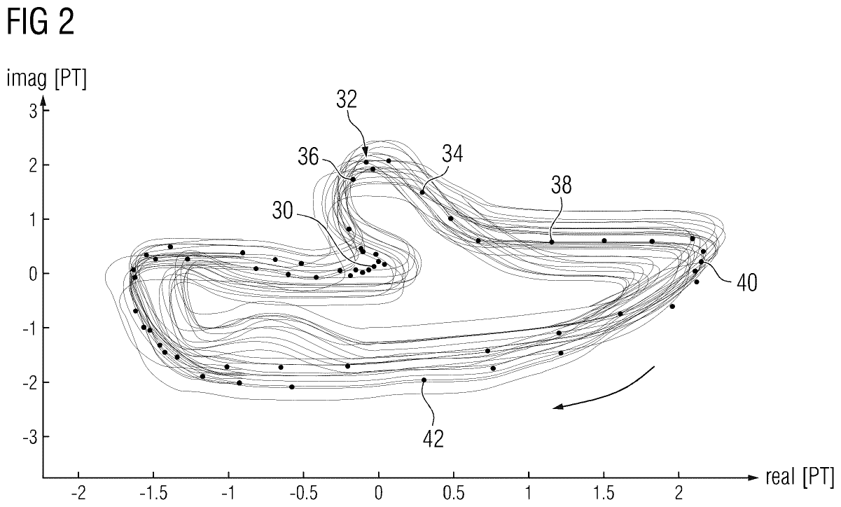

FIG. 2 depicts a two-dimensional PT signal plotted over multiple heart cycles in the complex plane. For example, FIG. 2 shows the real part and the imaginary part of the time derivative of the first (e.g., strongest) cardiac component. Samples at a time resolution of 25 ms are plotted as small circles, and the direction of the signal is indicated by the arrow. The signal displays a repetitive two-dimensional pattern with a well-defined change of direction 30 at about the end of the diastole (0,0). This is followed by a well-defined “notch” 32. The time point of the R-wave is indicated at 34. Taking into account the time delay of about 100 ms caused by the low pass filter delay, it is possible to set a trigger point at or before the R-wave (e.g., by triggering at the distinctive change of direction at point 30), or somewhat afterwards during the also distinctive notch 32. For example, setting the trigger point 36 will result in a trigger precisely at the R-wave. By contrast, the currently used trigger point is at 38, and thus, well after the R-wave at about 40% maximum contraction speed around (1,0). Therefore, by including the second cardiac component, earlier trigger time points 30 or 36 may be realized. Point 40 indicates the mid-systolic point of maximum contraction speed, and point 42 indicates the end-systole, where the contraction is maximal.

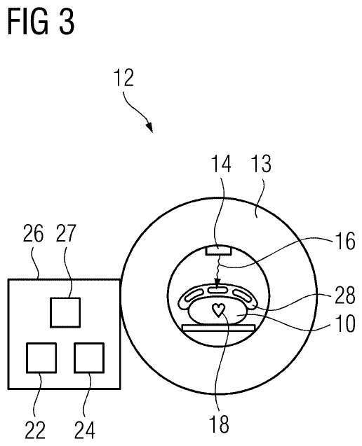

FIG. 3 finally shows a schematic MR scanner 12. The MR-scanner 12 includes a main magnet 13 and a control unit 24, by which the data acquisition of the MR scanner 12 may be controlled. The control unit 24 may be part of a computer device 26. The computer device 26 may also include a digital storage medium 22 and a user interface 27 including, for example, a display, a keyboard, mouse, a touch screen, or such like. A patient 10 may be examined, for example, in order to perform MR imaging of the heart 18.

A pilot tone signal 16 is emitted by a pilot tone emitter 14, which may be a separate RF source. In one embodiment, the pilot tone emitter 14 is positioned close to the heart (e.g., strapped to the local coil 28 or included in the coil). The pilot tone signal is modulated by the movement of the heart 18 and the lung (not shown). The (modulated) pilot tone signal is received by the receiver coil arrangement 28, which may be a local coil 28, such as an anterior (spine) coil, a head coil or chest array coil, but may also be the body coil, possibly including anterior (spine) coils.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for acquiring image data in a radiological examination of a part of a human or animal body, wherein the part is subjected to a cardiac movement, the method comprising:
transmitting a radiofrequency Tx Pilot Tone signal via at least one RF transmit antenna;
receiving a Pilot Tone signal from the body part via a radiofrequency receiver coil arrangement comprising a number of channels, wherein the received Pilot Tone signal comprises a number of channel signals associated with the number of channels;
carrying out a blind source separation algorithm on a training portion of the Pilot Tone signal and thereby determining weighting vectors to extract cardiac movement signals in the presence of other signals, wherein the weighting vectors allow to form weighted combinations of the number of channel signals;
selecting and storing at least two non-parallel weighting vectors of the weighting vectors that allow to extract signal components that represent cardiac movement from the number of channel signals;
applying the weighting vectors to further portions of the Pilot Tone signal to obtain a multi-dimensional Pilot Tone signal representing the cardiac movement, wherein the multi-dimensional Pilot Tone signal has at least two dimensions;
using the multi-dimensional Pilot Tone signal for controlling acquisition of image data or for retrospectively gating or correcting the acquired image data.

2. The method of claim 1, wherein the multi-dimensional Pilot Tone signal representing the cardiac movement has between two and five dimensions.

3. The method of claim 2, wherein the multi-dimensional Pilot Tone signal representing the cardiac movement has two or three dimensions.

4. The method of claim 1, wherein the blind source separation algorithm utilizes one or more Principal Component Analysis operations.

5. The method of claim 1, wherein the blind source separation algorithm utilizes one or more Independent Component Analysis operations.

6. The method of claim 1, wherein the blind source separation algorithm is used to detect a strongest independent component corresponding to the cardiac movement, and
wherein the method further comprises:
using a strongest independent component to retrospectively analyze the training portion of the Pilot Tone signal; and
detecting at least one further independent component corresponding to the cardiac movement from the retrospective analysis.

7. The method of claim 6, wherein using the strongest independent component to retrospectively analyze the training portion of the Pilot Tone signal comprises using the strongest independent component to retrospectively analyze the training portion of the Pilot Tone signal to average the Pilot Tone signal over a plurality of cardiac intervals, the plurality of cardiac intervals having been determined from the strongest independent component.

8. The method of claim 1, wherein the number of channel signals of the received Pilot Tone signal are complex-valued, and
wherein the weighting vectors each extract a real or an imaginary part of a signal component.

9. The method of claim 1, wherein the number of channel signals of the received Pilot Tone signal are complex-valued, and
wherein the number of channel signals are rotated in the complex plane before carrying out the blind source separation algorithm.

10. The method of claim 9, wherein the number of channel signals are rotated in the complex plane so that a mean of a rotated signal lies on one diagonal of the complex plane.

11. The method of claim 1, wherein the number of channel signals of the received Pilot Tone signal are complex, and the blind source separation algorithm is real-valued, and
wherein the method further comprises:
generating a real-valued matrix in which the real and imaginary parts of the complex channel signals form separate channels; and
performing a blind source separation algorithm on the real-valued matrix.

12. The method of claim 1, wherein a time derivative of the multi-dimensional Pilot Tone signal is used for controlling the acquisition of the image data.

13. The method of claim 1, further comprising determining trigger time points for triggering the acquisition of the image data, the determining of the trigger time points comprising evaluating properties of the multi-dimensional Pilot Tone signal or of a time derivative of the multi-dimensional Pilot Tone signal.

14. The method of claim 13, wherein evaluating properties of the multi-dimensional Pilot Tone signal or of the time derivative of the multi-dimensional Pilot Tone signal comprises evaluating position, direction, velocity, acceleration, change in direction in multi-dimensional signal space, or any combination thereof of the multi-dimensional Pilot Tone signal or the time derivative of the multi-dimensional Pilot Tone signal.

15. The method of claim 1, wherein the acquisition of the image data is triggered at a defined time point within a cardiac cycle.

16. The method of claim 15, wherein the defined time point is a time point between 250 ms before and 50 ms after an R-wave, between 200 ms before the R-wave and the R-wave, or between 150 ms and 20 ms before an R-wave.

17. The method of claim 1, further comprising applying an adaptive, stochastic, or model-based filter to the multi-dimensional Pilot Tone signal representing the cardiac movement, such that a filtered movement signal is obtained.

18. The method of claim 17, wherein the adaptive, stochastic, or model-based filter is adapted to obtain properties of the multi-dimensional Pilot Tone signal, the properties of the multi-dimensional Pilot Tone signal being a velocity vector or an acceleration vector.

19. A control unit comprising:
a processor configured to acquire image data in a radiological examination of a part of a human or animal body, wherein the part is subjected to a cardiac movement, the processor being configured to acquire the image data comprising the processor being configured to:
transmit a radiofrequency Tx Pilot Tone signal via at least one RF transmit antenna;
receive a Pilot Tone signal from the body part via a radiofrequency receiver coil arrangement comprising a number of channels, wherein the received Pilot Tone signal comprises a number of channel signals associated with the number of channels;
carry out a blind source separation algorithm on a training portion of the Pilot Tone signal and thereby determine weighting vectors to extract cardiac movement signals in the presence of other signals, wherein the weighting vectors allow to form weighted combinations of the number of channel signals;
select and store at least two non-parallel weighting vectors of the weighting vectors that allow to extract signal components that represent cardiac movement from the number of channel signals;
apply the weighting vectors to further portions of the Pilot Tone signal to obtain a multi-dimensional Pilot Tone signal representing the cardiac movement, wherein the multi-dimensional Pilot Tone signal has at least two dimensions;
use the multi-dimensional Pilot Tone signal for control of acquisition of the image data or for retrospective gate or correction of the acquired image data,
wherein the control unit is part of a radiological imaging modality.

20. The control unit of claim 19, wherein the radiological imaging modality comprises a magnetic resonance system.
